(19) United States
(12) Patent Application Publication
(10) Pub. No.: US 2018/0064342 A1
(43) Pub. Date: Mar. 8, 2018

(54) SYSTEM AND METHOD FOR ENHANCED MAGNETIC RESONANCE IMAGING OF TISSUE

(71) Applicant: IMRICOR MEDICAL SYSTEMS, INC., BURNSVILLE, MN (US)

(72) Inventor: Steven R. Wedan, Savage, MN (US)

(21) Appl. No.: 15/558,055

(22) PCT Filed: Mar. 18, 2016
(86) PCT No.: PCT/US16/23071

§ 371 Date: Sep. 13, 2017

Related U.S. Application Data
(60) Provisional application No. 62/134,654, filed on Mar. 18, 2015.

(51) Int. Cl. A61B 5/00; A61B 5/055

(52) U.S. Cl. CPC A61B 5/0044; A61B 5/055; A61B 5/0037; A61B 5/02007; A61B 5/7285; A61B 2560/0266; A61B 5/7257

(57) ABSTRACT

A method for producing a magnetic resonance image of a subject tissue to identify a lesion or scar tissue thereon in provided. The method includes acquiring an initial three-dimensional image of the subject tissue by a computer-implemented MRI system; identifying the surface of the subject tissue by the computer-implemented MRI system; selecting one or more points on the surface of the subject tissue by the computer-implemented MRI system; and acquiring by the computer-implemented MRI system, a first two-dimensional image for at least one of the selected points that is substantially tangential to the surface of the selected point.

### BACKGROUND OF THE INVENTION

### 1. Field

The present invention relates to magnetic resonance imaging at a location within a volume of tissue in a manner that enhances the visibility of the tissue within the volume. In particular, the present invention utilizes multiple scan planes in various orientations to generate images that accurately identify ablation lesions or scar tissue on the walls of the heart.

### 2. Description of the Related Art

There are many clinical applications that can benefit from accurate and detailed medical images. One important value of MRI guided cardiac interventions is the ability to image thin tissues. In particular, it is important to be able to effectively visualize cardiac ablation lesions or scar tissue in the walls of the heart, such as the atrial walls, which may have a thickness of 1 mm to 1.5 mm.

To produce an image, the MRI apparatus supplies energy of a specific frequency and energy to atomic nuclei positioned within a constant magnetic field, to cause the atomic nuclei to release energy, and converts the energy released from the atomic nuclei to signals to enable the imaging of soft tissue, lesions, and scar tissue within a human body. When utilizing signals from an MRI to produce images, magnetic field gradients are employed. Typically, the region to be imaged has a sequence of measurement cycles applied in which the MR gradients vary according to the particular localization method being used. The resulting set of received MR signals are digitized and processed to reconstruct the image using techniques know to those of skill in the art. The measurement cycle used to acquire each MR signal is performed in accordance with a pulse sequence produced by the MRI. Clinically available MRI systems store a library of such pulse sequences that can be prescribed to meet the needs of many different clinical applications. In addition to providing clinical pulse sequences, research MRI systems also enable the development of new pulse sequences.

The ability to effectively image a lesion or scar on the thin atrial wall with magnetic resonance is dependent, in part, on the orientation of the scan plane. The scan plane is the two-dimensional plane that defines the slice that is displayed in the resultant image. If the scan plane is oriented transversely, such that it “cuts” through the thin tissue, a lesion or scar may only be represented by a few pixels in the image and may go undetected.

Thus, what is needed is an improved system and method wherein small lesions and/or scar tissue may be effectively detected, identified and diagnosed.

### SUMMARY OF THE INVENTION

The present invention overcomes the aforementioned drawbacks by providing a computer-assisted system and method for producing MR images from which small lesions and scars may be accurately identified in MR images.

The present invention provides a method for producing an MR image whereby the imaging or scan plane is tangential or near-tangential to a location of interest in the tissue. In doing so, small lesions and scar tissue comprise a greater number of pixels in the resultant image and may be more easily detected, identified and diagnosed.

In other aspects of the invention, a method for producing a magnetic resonance image of a subject tissue to identify a lesion or scar tissue thereon in provided. The method includes acquiring an initial three-dimensional image of the subject tissue by a computer-implemented MRI system; identifying the surface of the subject tissue by the computer-implemented MRI system; selecting one or more points on the surface of the subject tissue by the computer-implemented MRI system; and acquiring by the computer-implemented MRI system, a first two-dimensional image for at least one of the selected points that is substantially tangential to the surface of the selected point.

The method may further include acquiring by the computer-implemented MRI system, one or more additional two-dimensional images for the selected point in scan planes that are oriented parallel to the first scan plane or in scan planes that are oriented in a plane other than parallel to the surface of the selected point.

In other aspects of the invention, the method includes using the one or more two-dimensional images to identify lesions or scar tissue.

In other aspects of the invention acquiring an initial three-dimensional image is accomplished by volume imaging.

In other aspects of the invention acquiring an initial three-dimensional image is generated from one or more two-dimensional images oriented in a plane parallel to each other, the one or more two-dimensional images comprising a set.

In yet other aspects of the invention each set of parallel two-dimensional images are orthogonally offset from each other.

In yet other aspects of the invention identifying a subject surface of interest is accomplished by pre-programmed software or by inputting into a computer having a knowledge base, surface identification parameters selected by a user.

In other aspects of the invention selecting multiple points on the surface of the subject tissue is done by pre-programmed software or inputting multiple user-selected points into a computer having a display module.

In yet other aspects of the invention creating a one or more two-dimensional images for each point selected further comprises acquiring additional two-dimensional images at each point, the additional images having a different scan plane orientation than substantially parallel or tangential to the surface at that point. This dithering helps to minimize the effect of encountering small variations in surface shape around the selected points and also allows for parallel imaging planes to be placed throughout the volume of interest within the tissue at small spatial intervals. This in turn allows for accurate identification of lesions or scar tissue within the volume of interest.

In other aspects of the invention the scan planes that are oriented in a plane other than parallel to the surface of the selected point of interest are oriented orthogonally.

In other aspects of the invention the scan planes that are oriented in a plane other than parallel to the surface of said selected point are oriented from substantially parallel to the surface of the selected point to orthogonal to the selected point.

These and other aspects of the invention will now be described with reference to the accompanying figures.

### BRIEF DESCRIPTION OF THE DRAWINGS

FIG. 1 is an illustration of a typical MRI system and its component parts.

FIG. 2 depicts a conventional manner of imaging a lesion on thin tissue, such as heart tissue, wherein a transverse scan plane produces an image wherein the lesion is represented by a few pixels.

FIG. 3 depicts imaging a lesion on thin tissue using a tangential scan plane in accordance with one aspect the invention.

FIG. 4 depicts forming an initial three-dimensional image utilizing three orthogonal scan sets.

FIG. 5 depicts imaging a surface utilizing parallel scan planes.

### DETAILED DESCRIPTION OF THE INVENTION

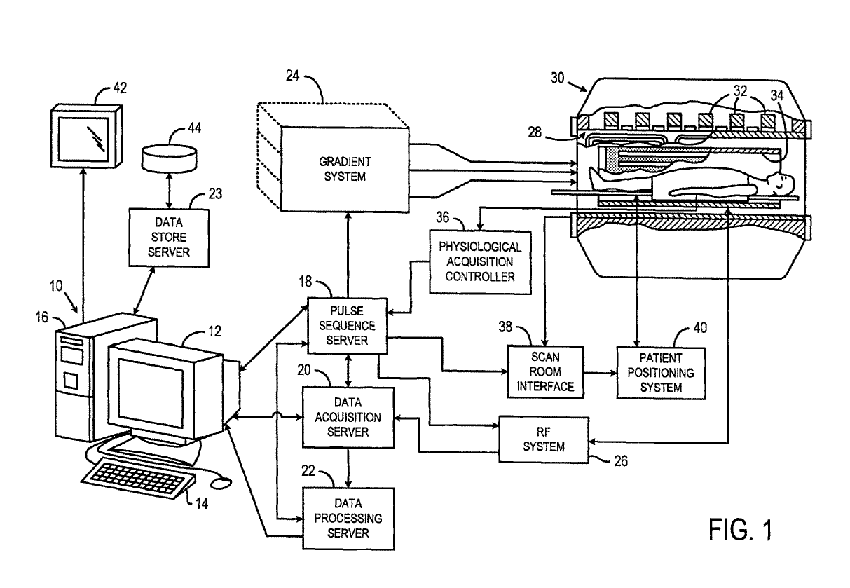

Referring now to FIG. 1, an MRI system includes a workstation 10 having a display 12 and a keyboard 14. The workstation 10 includes a processor 16 that is a commercially available programmable machine running a commercially available operating system. The workstation 10 provides the operator interface that enables scan prescriptions to be entered into the MRI system. The workstation 10 is coupled to four servers: a pulse sequence server 18; a data acquisition server 20; a data processing server 22, and a data store server 23. The workstation 10 and each server 18, 20, 22 and 23 are connected to communicate with each other.

The pulse sequence server 18 functions in response to instructions from the workstation 10 to operate a gradient system 24 and an RF system 26. Gradient waveforms necessary to perform the prescribed scan are produced and applied to the gradient system 24 that excites gradient coils in an assembly 28 to produce the magnetic field gradients used for position encoding MR signals. The gradient coil assembly 28 forms part of a magnet assembly 30 that includes a polarizing magnet 32 and a whole-body RF coil 34.

RF excitation waveforms are applied to the RF coil 34 by the RF system 26 to perform the prescribed magnetic resonance pulse sequence. Responsive MR signals detected by the RF coil 34 or a separate local coil (not shown in FIG. 1) are received by the RF system 26, amplified, demodulated, filtered and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase and pulse amplitude waveform. The generated RF pulses may be applied to the whole body RF coil 34 or to one or more local coils or coil arrays (not shown in FIG. 1).

The RF system 26 also includes one or more RF receiver channels. Each RF receiver channel includes an RF amplifier that amplifies the MR signal received by the coil to which it is connected and a detector that detects and digitizes the received MR signal.

The pulse sequence server 18 may also optionally receive patient data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heart beat.

The pulse sequence server 18 also connects to a scan room interface circuit 38 that receives signals from various sensors associated with the condition of the patient and the magnet system. It is also through the scan room interface circuit 38 that a patient positioning system 40 receives commands to move the patient to desired positions during the scan.

The digitized MR signal samples produced by the RF system 26 are received by the data acquisition server 20. The data acquisition server 20 operates in response to instructions downloaded from the workstation 10 to receive the real-time MR data and provide buffer storage such that no data is lost by data overrun. In some scans the data acquisition server 20 does little more than pass the acquired MR data to the data processor server 22. However, in scans that require information derived from acquired MR data to control the further performance of the scan, the data acquisition server 20 is programmed to produce such information and convey it to the pulse sequence server 18.

The data processing server 22 receives MR data from the data acquisition server 20 and processes it in accordance with instructions downloaded from the workstation 10. Such processing may include, for example Fourier transformation of raw k-space MR data to produce two or three-dimensional images.

Images reconstructed by the data processing server 22 are conveyed back to the workstation 10 where they are stored. Real-time images are stored in a data base memory cache (not shown) from which they may be output to operator display 12 or a display that is located near the magnet assembly 30 for use by attending physicians. Batch mode images or selected real time images are stored in a host database on disc storage 44. When such images have been reconstructed and transferred to storage, the data processing server 22 notifies the data store server 23 on the workstation 10. The workstation 10 may be used by an operator to archive the images, produce films, or send the images via a network to other facilities.

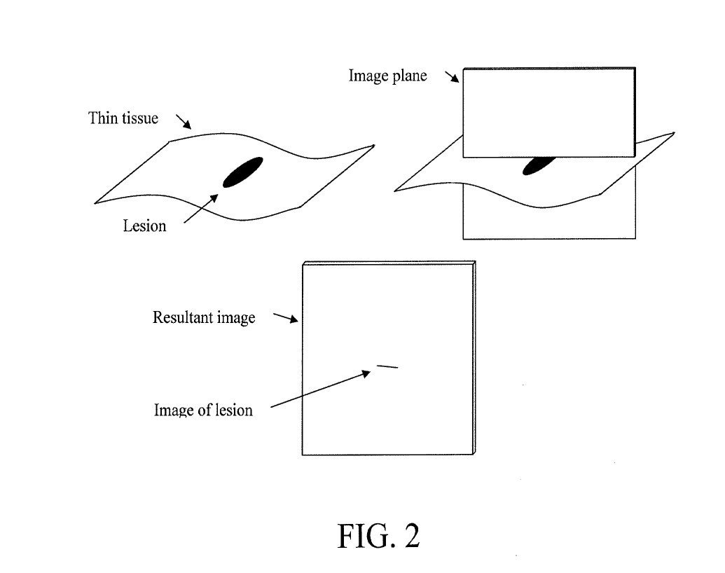

Referring now to FIG. 2 the acquisition of a conventional scan is depicted. The image plane is transverse to the tissue being imaged and results in an image with the lesion depicted by only a few pixels.

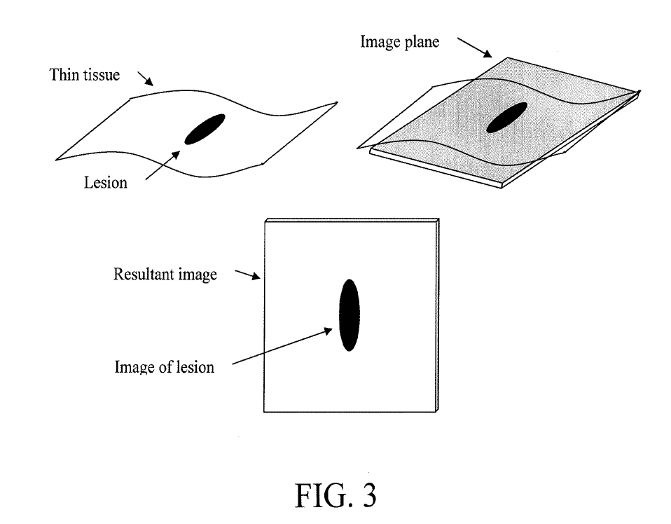

Referring now to FIG. 3 imaging a lesion on thin tissue using a tangential scan plane in accordance with the invention is depicted. FIG. 3 illustrates how using a tangential scan plane results in a MRI image in which a more readily identifiable lesion can be visualized due to the greater number of imaged pixels within the lesion. By way of example, the location of a catheter being used during an interventional procedure, such as ablation, is determined by MRI. After the ablation point is registered, software is used to identify a first plane tangential to the point of ablation. Subsequent two-dimensional images may be generated that have planes that are parallel to the first plane.

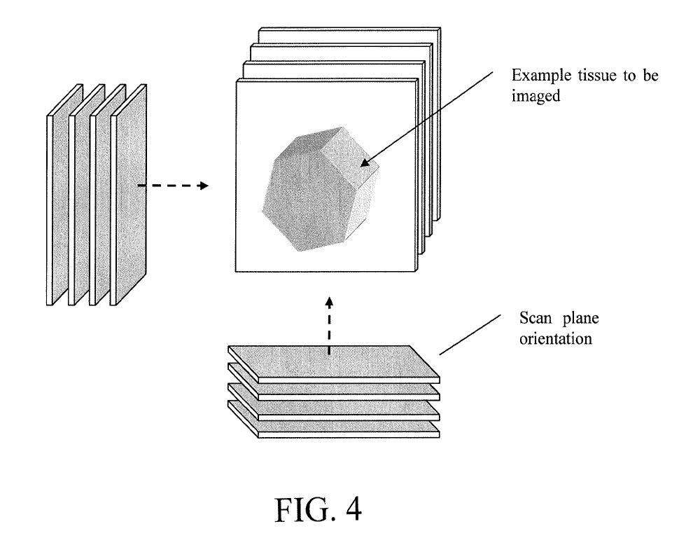

Referring now to FIG. 4 an initial three-dimensional MRI image may be created from a two-dimensional image set or by combining a plurality of two-dimensional image sets. Each set of two-dimensional images includes scan planes that are parallel to each other. Each set of parallel scan plane images are offset from each other. The illustration in FIG. 4 shows three orthogonal scan sets being used to form the initial three-dimensional image. Each set of imaging planes will produce a three-dimensional volume image, formed from the two-dimensional images oriented as shown. Other oblique imaging plane sets may also be used, oriented at any angle. Each individual three-dimensional volume may be combined with the others to render a high quality initial three-dimensional volume image. However, forming a three-dimensional image of the heart by using stacks of two-dimensional images means that the majority of the heart wall will only be visible as a cross-section of the heart and the information is limited to the two-dimensional planes. Although a three-dimensional image can be created, the additional information in the three-dimensional image is extracted from (or interpolated between) the two-dimensional slices. Because most organs, such as the heart, comprise closed surfaces very few of the two-dimensional planes will be oriented tangential to the tissue surface. To clearly identify a lesion on the surface of the tissue, such as the wall of a heart, additional steps of acquiring information are necessary.

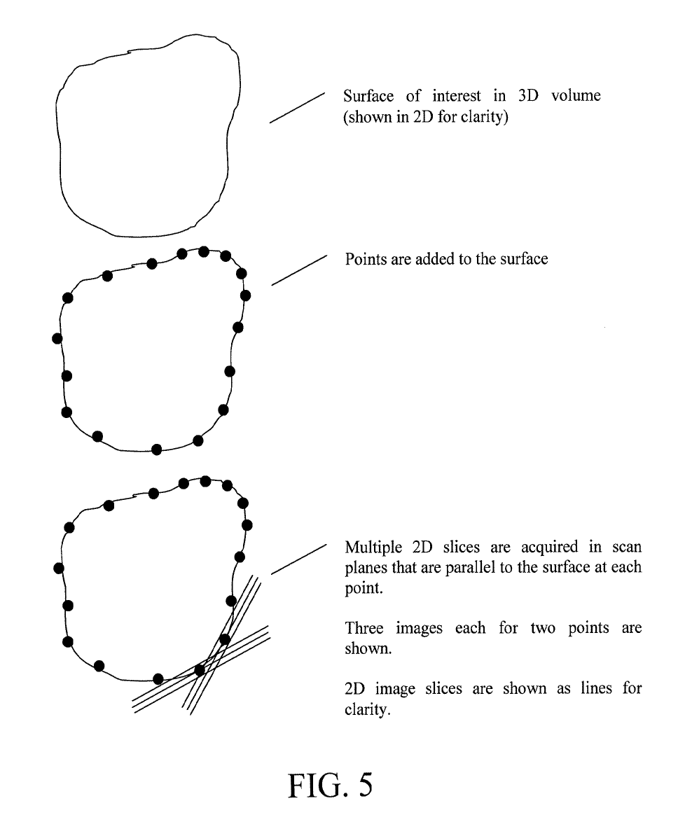

Referring now to FIG. 5 an “enhanced” three-dimensional image may be formed using MRI volume imaging (where the entire volume, such as the heart, is excited by the MRI) or by one or more sets of two-dimensional slices, each set having a unique scan plane orientation. As illustrated multiple two-dimensional slices are acquired in scan planes that are parallel to the surface at each point.

After a three-dimensional image is rendered, subsequent imaging planes may be oriented such that the tissue is optimally imaged, by orienting the new scan planes parallel to the surface of the tissue of interest. These new scan planes may or may not image the entire three-dimensional volume. They may, for instance, be restricted to locations in space near and/or including the tissue of interest. In addition, a first scan plane can be acquired that is parallel to the point of interest while subsequent scan planes may be acquired that are orthogonal to the first scan plane.

Each of the foregoing cases, i.e. using a set of parallel scan planes or a set of one parallel and several orthogonal scan planes, may be used independently to identify features of interest or to “enhance” the initial, three-dimensional image to better image lesions and/or scar tissue on the wall of the heart.

The following disclosure uses a cardiac example for illustration. However, those of skill in the art will appreciate that the present invention may be used with respect to any tissue where enhanced imaging by selective orientation of scan planes at various regions is desirable.

In a subject patient, ablation lesions exist on the thin atrial wall. A three-dimensional volume image of the patient's heart is imaged with MRI. Image processing software on a computer identifies and locates the surface of the atrium. Next, either automatically by referring to preregistered (or pre-programmed) regions within the heart in a stored knowledge base, or in response to user input, a plurality of new images are acquired, each image plane is oriented such that it is substantially parallel to the atrial wall. A particular region of atrial wall may be imaged by several parallel imaging planes. For example, one imaging plane may be outside the heart, one may include the heart wall, and one may be inside the heart. These three images would form a volume with enhanced visibility of that region of atrial wall. Those of skill in the art will appreciate that the number of slices needed to form the desired image may vary. In addition, several regions of atrial wall may be imaged as described, each using scan planes that are substantially parallel to the region of interest. In this way, enhanced MRI imaging of a portion or all of the atrial wall is achieved.

It should be appreciated that many equivalents, alternatives, variations, and modifications, aside from those expressly stated herein, are possible and are considered to be within the scope of the invention. Therefore, the invention should not be limited to any particular disclosed embodiment.

What is claimed is:

1. A method for producing a magnetic resonance image of a subject tissue to identify a lesion or scar tissue thereon comprising:
acquiring an initial three-dimensional image of said subject tissue by a computer-implemented MRI system;
identifying the surface of said subject tissue by the computer-implemented MRI system;
selecting one or more points on the surface of the subject tissue by the computer-implemented MRI system; and
acquiring by the computer-implemented MRI system, a first two-dimensional image for at least one of said selected points that is substantially tangential to the surface of said selected point.

2. The method of claim 1 further comprising acquiring by the computer-implemented MRI system, one or more additional two-dimensional images for said selected point in scan planes that are oriented parallel to said first scan plane or in scan planes that are oriented in a plane other than parallel to the surface of said selected point.

3. The method of claim 1 further comprising using said plurality of two-dimensional images to enhance the initial three-dimensional image to identify lesions or scar tissue.

4. The method of claim 1 wherein acquiring an initial three-dimensional image is accomplished by volume imaging.

5. The method of claim 1 wherein acquiring an initial three-dimensional image is generated from one or more two-dimensional images oriented in a plane parallel to each other, the one or more two-dimensional images comprising a set.

6. The method of claim 5 wherein each set of one or more parallel two-dimension images are orthogonally offset from each other.

7. The method of claim 1 wherein identifying a subject surface of interest is accomplished by pre-programmed software or inputting into a computer surface identification parameters selected by a user.

8. The method of claim 1 wherein selecting multiple points on the surface of the subject tissue is done by pre-programmed software having a knowledge base.

9. The method of claim 1 wherein selecting multiple points on the surface of the subject tissue is done by inputting multiple points into the computer by a user.

10. The method of claim 1 wherein said scan planes that are oriented in a plane other than parallel to the surface of said selected point are oriented orthogonally.

11. The method of claim 1 wherein said scan planes that are oriented in a plane other than parallel to the surface of said selected point are oriented from substantially parallel to the surface of said selected point to orthogonal to said selected point.
